(19) United States
(12) Patent Application Publication
(10) Pub. No.: US 2004/0186343 A1
(43) Pub. Date: Sep. 23, 2004

(54) Sexually stimulating garment

(76) Inventor: Robin B. Moore-Steele, Meridian, MS (US)

Correspondence Address: Stephen R. Greiner, Esquire, GREINER LAW OFFICES, P.C., Suite 110, 6701 Democracy Blvd., Bethesda, MD, 20817, US

(21) Appl. No.: 10/388,532

(22) Filed: Mar. 17, 2003

(51) Int. Cl. A61F 5/00; A41B 9/04

(52) U.S. Cl. 600/38; 2/406

(57) ABSTRACT

A garment for sexually stimulating a female wearer. The garment includes at least one stimulating member secured within the crotch portion thereof. The stimulating member is disposed so as to squeeze the labia minus of a wearer.

### FIELD OF THE INVENTION

The present invention relates generally to articles of apparel particular to a female wearer. BACKGROUND OF THE INVENTION

Apparatus for massaging the genitals of a female for therapeutic or erotic purposes are well known. Many of these apparatus are electrically powered or require the constant attention of the hands of a user to obtain the desired effect. Of course, being removed from an electrical current source or being unable to use one's hands can cause difficulties in using some massaging apparatus. A need presently exists, therefore, for a manual device for massaging the genitals of a female user in a hands-free manner.

### SUMMARY OF THE INVENTION

In light of the problems associated with the known massaging apparatus, it is a principal object of the invention to provide a garment that can massage the genitals of a female wearer without bringing hands, electricity, or medications into play. The garment can be discretely worn and has stimulating means that cover the groin and extend over and around the genitals, perineum, and anus of a wearer. In use, portions of the stimulating means rub against the clitoris and labia during leg movement to cause sexual stimulation in a wearer and, perhaps, sexual satisfaction in her mate should circumstances permit.

It is another object of the invention to provide a stimulating garment that can take the form of innerwear as well as outerwear with and without legs.

It is another object of the invention to provide a garment of the type described that is easy and intuitive to use, requiring little, if any, instruction to employ.

It is a further object of the invention to provide a garment of the type described that can be sized so that women of varying height, weight, and stature can wear it. Thus, the garment can be universally employed.

It is an object of the invention to provide improved elements and arrangements thereof in a sexually stimulating garment for the purposes described which is lightweight in construction, inexpensive to manufacture, and fully dependable in use.

Briefly, the sexually stimulating garment in accordance with this invention achieves the intended objects by featuring a front part and rear part joined together so as to define a waist opening at the top thereof and a pair of adjacent leg openings at the bottom thereof. A coversheet is secured to the crotch portion of the front part between the leg openings. In one embodiment of the garment, a number of stimulating members are positioned between the coversheet and the front part. One pair of stimulating members is disposed so as to squeeze the labia minus of a wearer together and another one of the stimulating members is disposed so as to rub the clitoris the wearer.

The foregoing and other objects, features and advantages of the present invention will become readily apparent upon further review of the following detailed description of the preferred embodiment as illustrated in the accompanying drawings.

### BRIEF DESCRIPTION OF THE DRAWINGS

The present invention may be more readily described with reference to the accompanying drawings, in which:

FIG. 1 is a perspective view of sexually stimulating garment in accordance with the present invention.

FIG. 2 is a cross-sectional view of the sexually stimulating garment taken along line 2—2 of FIG. 1.

FIG. 3 is a diagrammatic view showing the positioning of the stimulating members of the garment of FIG. 1 when such are engaged with the body of a female user.

FIG. 4 is a cross-sectional view of the garment taken along line 4—4 of FIG. 1.

FIG. 5 is a front elevational view of a first, alternative, sexually stimulating garment.

FIG. 6 is a front elevational view of a second, alternative, sexually stimulating garment.

FIG. 7 is a front elevational view of a third, alternative, sexually stimulating garment.

FIG. 8 is a front elevational view of a fourth, alternative, sexually stimulating garment.

Similar reference characters denote corresponding features consistently throughout the accompanying drawings.

### DETAILED DESCRIPTION OF THE PREFERRED EMBODIMENTS

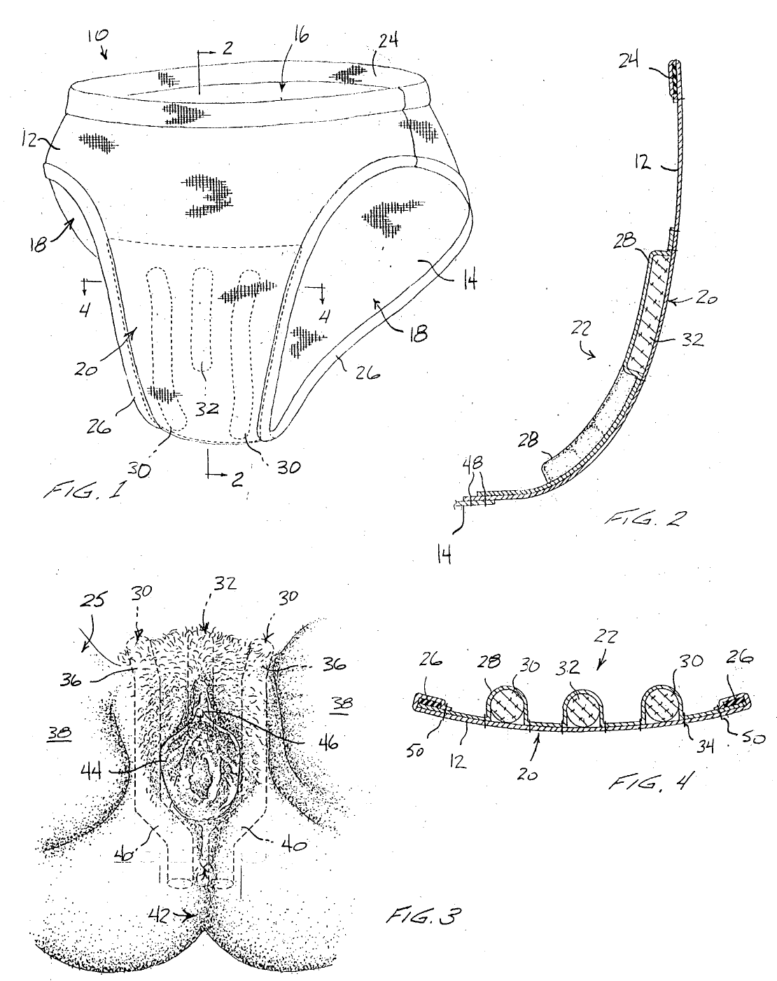

Referring now to FIGS. 1-4, a sexually stimulating garment in accordance with the present invention is shown at 10. Garment 10 includes a front part 12 and a rear part 14 joined at their corners so as to define panties with a waist opening 16 in the top thereof and a pair of crotch portion 20 to which are secured means 22 for sexually stimulating a wearer 25 of garment 10.

Parts 12 and 14 are triangular pieces of cloth that are sewn in registry to one another. An elastomeric waistband 24 is hemmed to the top edges of parts 12 and 14 and a pair of elastomeric leg bands 26 are hemmed to the bottom edges of parts 12 and 14. Bands 24 and 26 serve to reinforce parts 12 and 14 and, also, to retain garment 10 and stimulating means 22 snugly against the body of a wearer 25 during use.

Stimulating means 22 comprise a cloth coversheet 28, dimensioned to fit atop crotch portion 20 without overlapping into leg openings 18, having a number of stimulating members 30 and 32 secured by stitching 34 to the bottom thereof. Preferably, members 30 and 32 are short pieces of cotton cording. Members 30 and 32 can, alternatively, be formed of foam rubber or even a low density/high loft material like cotton wadding or wood pulp fiber. Low density/high loft materials, of course, offer the capability of absorbing and distributing body fluids.

The dimensions of stimulating members 30 and 32 are important to obtain the maximum stimulating effect. Members 30 and 32 have a diameter of about 0.4 inches (1.0 cm) and lengths of 6.0 inches (15.0 cm) and 2.4 inches (6.0 cm) respectively. Members 30 and 32 are spaced about 0.5 inches (1.25 cm) apart in a generally parallel arrangement. The top ends 36 of members 30, however, angle outwardly from one another to follow the contours of the upper thighs 38 of wearer 25. Further, the bottom ends 40 of members 30 angle inwardly toward one another to enter the anal cleft 42 of wearer 25. In use, members 30 squeeze the labia minus 44 and member 32 rubs the clitoris 46 of wearer 25 during movement of her thighs 38 to cause sexual stimulation in wearer 25.

Garment 10 can be made with conventional equipment in a continuous production process. First, parts 12 and 14, bands 24 and 26, coversheet 28 and stimulating members 30 and 32 are cut from stock material. Next, members 30 and 32 are positioned against coversheet 28. Then, the bottoms of coversheet 28 and members 30 and 32 are overlapped upon bottoms of parts 12 and 14, and all three elements are sewn together by bottom seams 48. Subsequently, the top of coversheet 28 and the sides thereof are sewn to crotch portion 20 with the sides being retained by the elastomeric leg band retaining hems 50. After sewing waistband 24 to parts 12 and 14, garment 10 is ready for wear. Of course, other processes can be used to construct garment 10.

Regardless of the method of construction employed, crotch portion 20 of garment 10 has an overall thickness of about 0.5 inches (1.25 cm) and is capable of bending to conform to the body of a wearer and provide a stimulating effect as the wearer moves about or even sits. Due to the unique configuration of stimulating members, garment 10 is unobtrusive even when worn beneath a tight-fitting outer garment. After use, garment 10 can be easily doffed and washed with a conventional detergent for reuse at a later time.

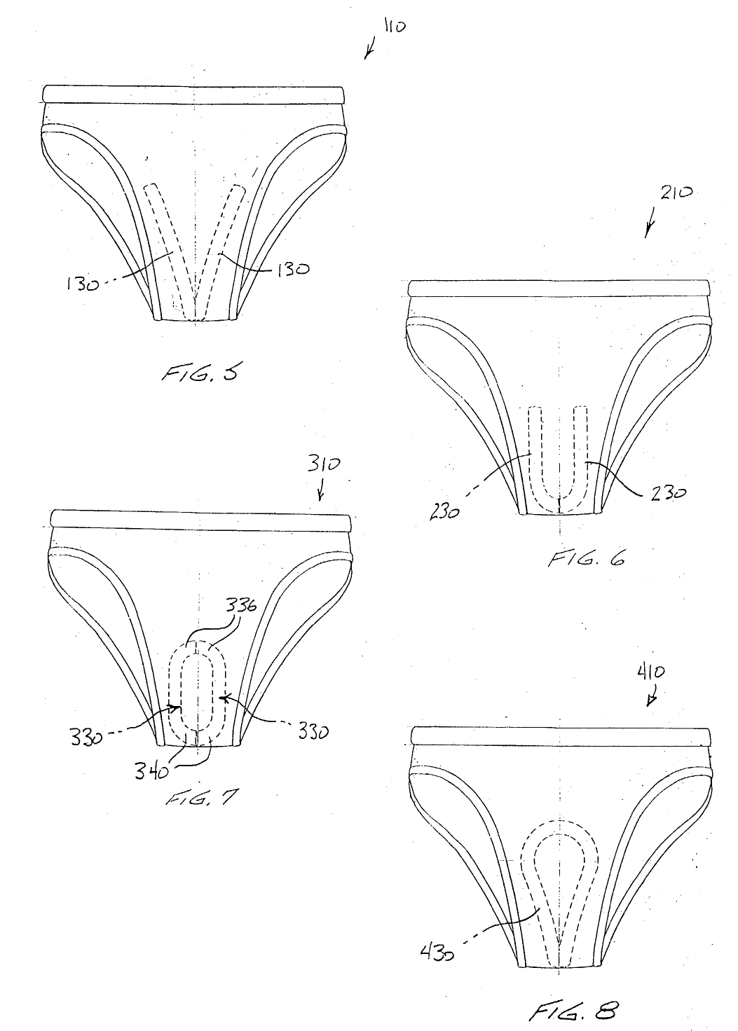

Experimentation with garment 10 has indicated that the configuration of members 30 and 32 can be modified to provide a heightened stimulating effect to some wearers. For example, some wearers prefer garments with member 32 removed. Others, however, fancy garments 110 and 210 wherein their respective stimulating members 130 and 230 provide geometric shapes that are closed at their its bottom ends like the V-shape shown in FIG. 5 and the U-shape in FIG. 6. Still others favor a garment 310 wherein the top ends 336 and the bottom ends 340 of members 330 are angled inwardly so as to contact one another as shown in FIG. 7. With ends 336 and 340 in contact, members 330 form a ring that can encircle the genitals of a female wearer. This ring (like any of the continuous geometric shapes of FIGS. 5, 6 and 7) can be formed from a single member 430 bent into an “inverted teardrop” shape as is illustrated in garment 410 of FIG. 8.

While the invention has been described with a high degree of particularity, it will be appreciated by those skilled in the art that modifications may be made thereto. For example, the invention has been illustrated for the sake of convenience in the form of panties, nonetheless, it can easily take the form of other articles of apparel with or without legs such as: pants, trousers, culottes, briefs, shorts, and G-strings. Therefore, it is to be understood that the present invention is not limited to the various embodiments described above, but encompasses any and all embodiments within the scope of the following claims.

I claim:

1. A sexually stimulating garment, comprising:
a front part, having a crotch portion, and a rear part joined to said front part so as to define a waist opening at the top thereof and a pair of adjacent leg openings at the bottom thereof; and,
a stimulating member secured to said crotch portion, and said stimulating member being disposed so as to squeeze the labia minus of a wearer.

2. The garment according to claim 1 wherein said stimulating member is a length of cording.

3. The garment according to claim 1 wherein said stimulating member is formed into a ring so as to engage said labia of a wearer from opposite sides.

4. The garment according to claim 1 wherein said stimulating member is disposed in a U-shape so as to engage said labia of a wearer from opposite sides.

5. The garment according to claim 1 wherein said stimulating member is disposed in an inverted teardrop-shape so as to engage said labia of a wearer from opposite sides.

6. A sexually stimulating garment, comprising:
a front part, having a crotch portion, and a rear part joined to said front part so as to define a waist opening at the top thereof and a pair of adjacent leg openings at the bottom thereof on opposite sides of said crotch portion;
a coversheet secured to said crotch portion; and,
a pair of stimulating members positioned between said coversheet and said front part, and said stimulating member being disposed so as to squeeze the labia minus of a wearer.

7. The garment according to claim 6 wherein each of said stimulating members is a length of cording.

8. The garment according to claim 6 wherein said stimulating members are disposed substantially parallel to one another.

9. The sexually stimulating garment according to claim 6 wherein the top ends of said stimulating members angle outwardly from one another and the bottom ends of said stimulating members angle inwardly toward one another.

10. The sexually stimulating garment according to claim 6 further comprising a third stimulating member positioned between said pair of stimulating members for rubbing the clitoris of a wearer.

11. The sexually stimulating garment according to claim 6 wherein said pair of stimulating members is disposed in a V-shape.

12. A sexually stimulating garment, comprising:
a front part, having a crotch portion, and a rear part joined to said front part so as to define a waist opening at the top thereof and a pair of adjacent leg openings at the bottom thereof on opposite sides of said crotch portion;
a coversheet secured to said crotch portion; and,
a plurality of stimulating members positioned between said coversheet and said front part, a pair of said stimulating members being disposed so as to squeeze the labia minus of a wearer together and another of said stimulating members being disposed so as to rub the clitoris the wearer.

13. The garment according to claim 12 wherein one pair of said stimulating members is disposed on opposite sides of said coversheet and each has a first length and another of said stimulating members is disposed between said pair of said stimulating members and has a second length that is less than said first length.

14. The garment according to claim 13 wherein the top ends of said pair of said stimulating members angle outwardly from one another and the bottom ends of said pair of said stimulating members angle inwardly toward one another.
